(19) United States
(12) Patent Application Publication
(10) Pub. No.: US 2020/0013191 A1
(43) Pub. Date: Jan. 9, 2020

(54) ELECTRONIC SURGICAL NEEDLE COUNTING SYSTEMS AND METHODS

(71) Applicant: Christoper M. Berning, Redwood City, CA (US)

(72) Inventor: Christoper M. Berning, Redwood City, CA (US)

(21) Appl. No.: 16/029,813

(22) Filed: Jul. 9, 2018

(51) Int. Cl. G06T 7/77; A61B 50/24; A61B 90/96; A61B 50/33; G06K 7/14

(52) U.S. Cl. CPC G06T 7/77; A61B 50/24; A61B 2050/21; A61B 50/33; G06K 7/1413; A61B 90/96

(57) ABSTRACT

The disclosure includes a surgical needle counting system comprising a stand, a camera, a touch screen device, a processor system, and a memory. The memory may include executable instructions that, when executed by the processor system, cause the processor system to effectuate operations. The operations may include receiving a first input comprising at least one of a style, dimension, and an initial quantity of at least one surgical needle prior to the at least one surgical needle entering a surgical field. The operations may comprise capturing an image of the at least one surgical needle after at least one of the at least one surgical needle has exited the surgical field. The operations may include analyzing the image to determine a final quantity of the at least one surgical needle after exiting the surgical field. The operations may incorporate determining whether the initial quantity equals the final quantity.

### BACKGROUND

### Field

Various embodiments disclosed herein relate to electronic surgical needle counting systems and methods.

### Description of Related Art

There are many tools used during medical procedures, such as surgery. These tools may include surgical items, such as surgical needles, sponges, retractors, forceps, scalpels, clips, and clamps. Because of the complexity of procedures and number of surgical items used, it is not uncommon for one of those items to be left inside the patient after surgery. It is so common that medical professionals have introduced the vernacular “retained surgical items” to describe these events. Environmental settings can increase the likelihood of surgical bodies left in the patient after surgery, such as emergency and urgent procedures, or complications and unexpected changes during the procedure.

Retained surgical items can be an issue for patients, surgeons, hospitals, and the entire medical team, as they can have both medical and legal implications. They can have potentially harmful consequences for the patient, including additional surgeries, pain abscesses, perforations, punctures, obstructions, and death. This surgical equipment may remain in a patient for days, months, or even years before manifesting as inflammatory reactions. During this period, the retained surgical item may migrate to different parts of the body, potentially causing additional damage and putting the patient at increased risk of one of the many painful consequences. Retained surgical items can also have costly consequences for doctors and hospitals, such as legal costs, operating room time, and x-ray costs. While there are current controls in place in hospitals to prevent retained surgical bodies, human error can affect the success of these methods. Thus, there is a need for a more effective way to account for surgical bodies before, during, and after surgery.

### SUMMARY

The disclosure describes methods of using a surgical needle counting system. The surgical needle counting system may include a stand, a camera coupled to the stand, a touch screen device communicatively coupled to the camera, a processor system communicatively coupled to the camera, and memory communicatively coupled to the processor system. The memory may comprise executable instructions that, when executed by the processor system, cause the processor system to effectuate operations.

The operations may comprise receiving a first input that may include at least one of a style, dimension, and an initial quantity of at least one surgical needle prior to the at least one surgical needle entering the surgical field. The operations may also include capturing an image of the at least one surgical needle after at least one of the at least one surgical needle has exited the surgical field. The operations may also consist analyzing the image to determine a final quantity of the at least one surgical needle after the at least one of the at least one surgical needle has exited the surgical field. The operations may further include determining whether the initial quantity equals the final quantity.

The surgical needle counting system may include a bar code reader. The bar code reader may be coupled to the stand and communicatively coupled to the touch screen device. The stand may define a rectangular shape having a first sidewall, a second sidewall opposite the first sidewall, a third sidewall adjacent the first and second sidewalls, an open side opposite the third sidewall, a top portion, and a bottom portion opposite the top portion. The stand may be constructed of a translucent material. The touch screen device may be coupled to the top portion of the stand.

The surgical needle counting system may further comprise a tray arranged and configured to receive the at least one surgical needle. The tray may be translucent and may have an adhesive surface. The bottom portion of the stand may be sized and configured to receive a tray of surgical needles. The camera may be arranged and configured so that the camera is able to capture images of an entire surface of the tray of surgical needles. The surgical needle counting system may also include a backlight located along the bottom portion such that the backlight is arranged and configured to provide contrast between the at least one surgical needle and the tray.

The camera may be located adjacent the top portion of the stand and vertically located between the touch screen device and the bottom portion. The bar code reader may be located adjacent the top portion of the stand and vertically located between the touch screen device and the bottom portion. The stand may include a semi-enclosed portion vertically located between the top portion and the bottom portion. The camera and the bar code reader may be located within the semi-enclosed portion. The processor system and memory may be integrated into the touch screen device.

The surgical needle counting system may further comprise an IV pole clamp coupled to the stand. The IV pole clamp may be arranged and configured to couple the stand to an IV pole.

The disclosure describes a method of counting surgical needles using the surgical needle counting system. The surgical needle counting system may include a stand, a camera coupled to the stand, a touch screen device communicatively coupled to the camera, a processor system communicatively coupled to the camera, and memory communicatively coupled to the processor system.

The method may comprise capturing, via the camera, a first image of the at least one surgical needle after at least one of the at least one surgical needle has exited the surgical field. The method may also include analyzing, via the processor system, the first image to determine a final quantity of the at least one surgical needle after the at least one of the at least one surgical needle has exited the surgical field. The method may further include determining, via the processor system, whether the initial quantity equals the final quantity.

The surgical needle counting system may include a bar code reader coupled to the stand and communicatively coupled to the touch screen device. The method of counting surgical needles may further include receiving, via the bar code reader, a first input that may consist of at least one of a style, dimension, and an initial quantity of at least one surgical needle prior to the at least one surgical needle entering the surgical field.

The method of counting surgical needles may further comprise displaying, via the touch screen device, a notification regarding whether the initial quantity equals the final quantity. The initial quantity may be a first initial quantity. The method may further include receiving, via the bar code reader, a second input that may consist of at least one of the style, dimension, and a second initial quantity of the at least one surgical needle prior to the at least one surgical needle entering the surgical field. The method of counting surgical needles may also include capturing, via the camera, a second image of the at least one surgical needle after at least one of the at least one surgical needle has exited the surgical field.

The final quantity may be a first final quantity. The method may further include analyzing, via the processor system, the second image to determine a second final quantity of the at least one surgical needle after the at least one of the at least one surgical needle has exited the surgical field. The method may also include determining, via the processor system, whether a sum of the first initial quantity and the second initial quantity equals a sum of the first final quantity and the second final quantity.

The method of counting surgical needles may also consist of receiving, via the touch screen, a manual input that may comprise a manual final quantity of the at least one surgical needle while the at least one surgical needle is still located on the surgical field. The method may further include determining, via the processor system, whether the initial quantity equals a sum of the final quantity and the manual final quantity.

### BRIEF DESCRIPTION OF THE DRAWINGS

These and other features, aspects, and advantages are described below with reference to the drawings, which are intended to illustrate, but not to limit, the invention. In the drawings, like reference characters denote corresponding features consistently throughout similar embodiments.

FIG. 1 illustrates a perspective view of a surgical needle counting system, according to some embodiments.

FIG. 2 illustrates a perspective view of a surgical needle counting system, according to some embodiments.

FIG. 3A illustrates a perspective view of a surgical needle counting system, according to some embodiments.

FIGS. 3B and 3C illustrate a tray for holding surgical needles, according to some embodiments.

FIG. 4 illustrates a perspective view of a surgical needle counting system, according to some embodiments.

FIG. 5 illustrates a method of using a surgical needle counting system, according to some embodiments.

FIG. 6 illustrates a surgical needle counting system with a bar code reader, according to some embodiments.

FIG. 7 illustrates a perspective field of view of a camera of a surgical needle counting system, according to some embodiments.

FIG. 8 illustrates a perspective view of a surgical needle counting system with an IV pole clamp, according to some embodiments.

FIGS. 9-11 illustrate methods of using an electronic surgical needle counting system, according to some embodiments.

### DETAILED DESCRIPTION

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### REFERENCE NUMERALS

10—Surgical needle counting system
12—Stand
14—Camera
16—Touch screen device
18—Processor system
20—Memory
22—First input
24—Initial quantity
26—At least one surgical needle
28—Image
30—Final quantity
40—Bar code reader
42—First sidewall
44—Second sidewall
46—Third sidewall
48—Open side
50—Top portion
52—Bottom portion
60a—Tray
60b—Tray cover
62—Suture coupling surface
63—Numbered surface
64—Backlight
65—Overhead light
66—Semi-enclosed portion
67—Field of view
68—IV pole clamp
70—First image
72—Second input
74—First initial quantity
76—Second initial quantity
78—Second image
80—First final quantity
82—Second final quantity
84—Manual input
86—Manual final quantity

Surgical items mistakenly left in patients after surgery is a costly, but common, problem in hospitals. Current controls to prevent such occurrences oftentimes involve manual counting of each surgical item before and after surgery. Unfortunately, this method is prone to error, and in many cases results may come out correct even though a retained surgical item may be discovered at a later time.

This disclosure provides systems of accounting for surgical items in order to prevent such episodes. Such systems may thereby augment or fully replace current controls to prevent retained surgical items. Embodiments may be implemented in the form of an electronic surgical needle counting system 10 that can provide a controlled count of surgical items—not prone to human error. While the surgical needle counting system 10 is described for use with surgical needles, it should be appreciated that the system 10 can be used to account for any surgical item. Moreover, the system 10 can operate on any processing system available to people outside of the operating room in order to provide higher levels of control for medical and non-medical applications.

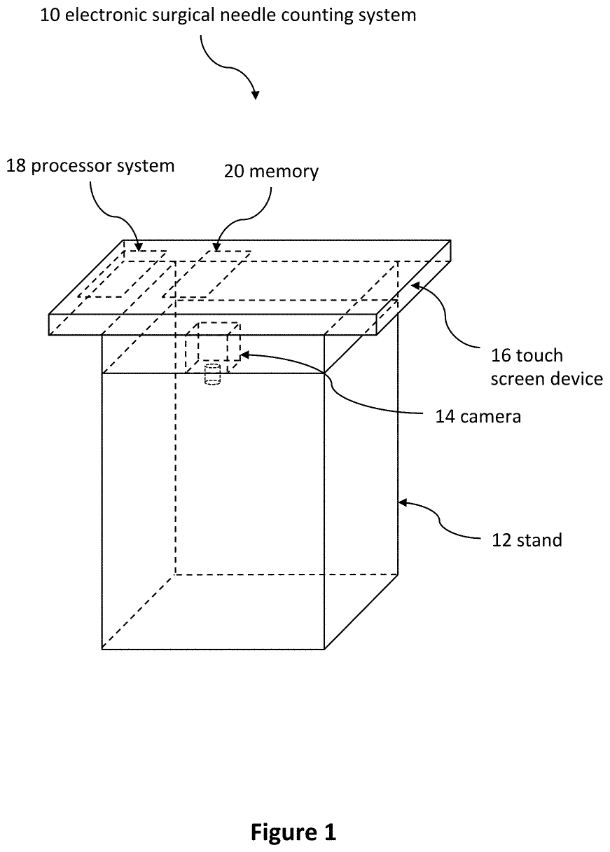

As illustrated in FIG. 1, the disclosure includes an electronic surgical needle counting system 10 (herein after referred to as “system 10”). In many embodiments, the system 10 may include a stand 12, a camera 14 coupled to the stand 12, a touch screen device 16 communicatively coupled to the camera 14, a processor system 18 communicatively coupled to the camera 14, and a memory 20 communicatively coupled to the processor system 18. The memory 20 may incorporate executable instructions that, when executed by the processor system 18, cause the processor system 18 to effectuate operations.

The operations may consist of receiving a first input 22 that may include at least one of a style, dimension, type, curvature, body, point, shape, length, color, an initial quantity 24, and the like of at least one surgical needle 26. The first input 22 may be received by the system 10 prior to the at least one surgical needle 26 entering a surgical field. The operations may also include capturing an image 28 of the at least one surgical needle 26. The image 28 may be captured by the system 10 after at least one of the at least one surgical needle 26 has exited the surgical field. The operations may also comprise analyzing the image 28 to determine a final quantity 30 of the at least one surgical needle 26 after the at least one of the at least one surgical needle 26 has exited the surgical field. Furthermore, the operations may include determining whether the initial quantity 24 equals the final quantity 30.

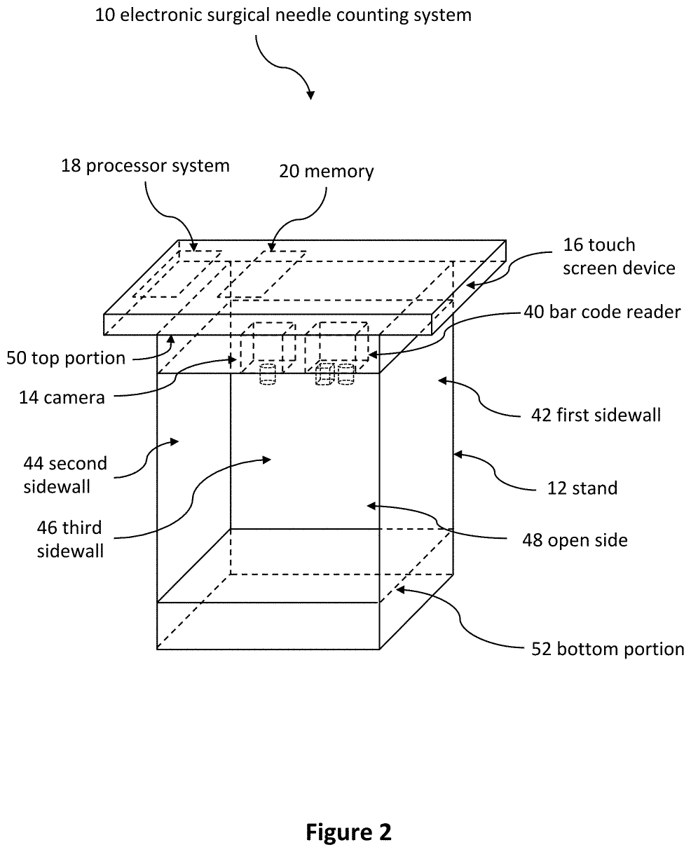

Referring now to FIG. 2, the system 10 may also include a bar code reader 40. The bar code reader 40 may be coupled to the stand 12 and communicatively coupled to the touch screen device 16. The bar code reader 40 may be configured to read at least one of one-dimensional barcodes, two-dimensional barcodes, and the like. The bar code reader 40 may be arranged and configured to read a variety of bar codes, including a UPC barcode, EAN barcode, ITF barcode, Codabar barcode, Modified Plessey barcode, QR code, Datamatrix code, and the like.

In some embodiments, the bar code reader 40 may be coupled to the camera 14. For example, the barcode reader 40 and the camera 14 may embody one device coupled to the system 10, such that the camera 14 may be arranged and configured to have the ability to capture the image 28 of the at least one surgical needle 26, as well as have the ability to capture an image of, and read a bar code.

In some embodiments, the stand 12 of the system 10 may define a rectangular shape. The stand 12 may comprise a first sidewall 42, a second sidewall 44 opposite the first sidewall 42, a third sidewall 46 adjacent the first sidewall 42 and the second sidewall 44, an open side 48 opposite the third sidewall 46, a top portion 50, and a bottom portion 52 opposite the top portion 50. In some embodiments, the stand 12 of the system 10 may consist of at least one sidewall. For example, the stand 12 may include at least one of only one sidewall and only two sidewalls, such that the stand 12 may receive surgical tools from multiple sides. In a further example, the stand may include four sidewalls, one of which may open to allow insertion of surgical tools for counting by the system 10.

It should be appreciated that the stand 12 of the system 10 may define any shape deemed appropriate by those skilled in the art, such as rectangular, circular, triangular, and the like. In some embodiments, the stand 12 may define a C-shape, such that the stand 12 may be constructed of one rounded sidewall with an opening through which surgical tools may be inserted for counting by the system 10.

In some embodiments, the stand 12 may be constructed of a translucent material. The translucent material may include at least one of plexiglass, glass, plastic, rubber, silicone, any plastic like material, and the like. The translucent material of the stand 12 may enable a user of the system 10 to view surgical items within the stand 12 being analyzed by the system 10. For example, the system 10 may be used in conjunction with current methods of accounting for surgical items such that the translucent material of the stand 12 may allow the user to conduct a count simultaneously with the system 10. In some embodiments, the stand 12 is comprised of at least one of translucent material, transparent material, opaque material, solid material, and the like.

With added reference to FIG. 2, the touch screen device 16 may be coupled to the top portion 50 of the stand 12. The touch screen device 16 may be coupled to the top portion 50 in a fashion that a user interface of the touch screen device 16 may be upward facing such that the user of the system 10 may utilize the touch screen device 16. In some embodiments, the touch screen device 16 may be coupled to one of the sidewalls of the stand 12, wherein the top portion 50 includes an additional wall. The additional wall composing the top portion 50 may stop anything from falling into the stand 12, as such items may disrupt the count.

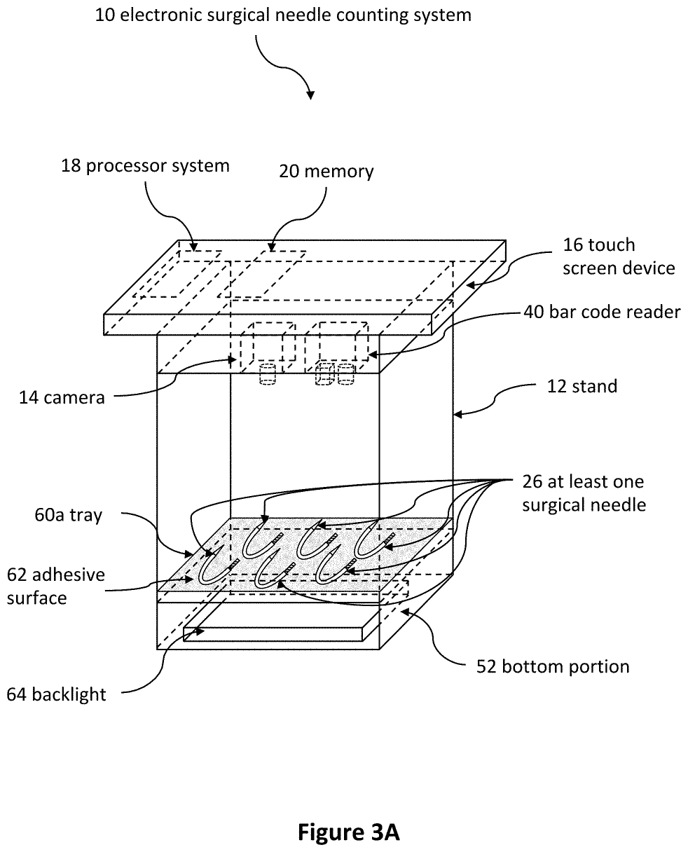

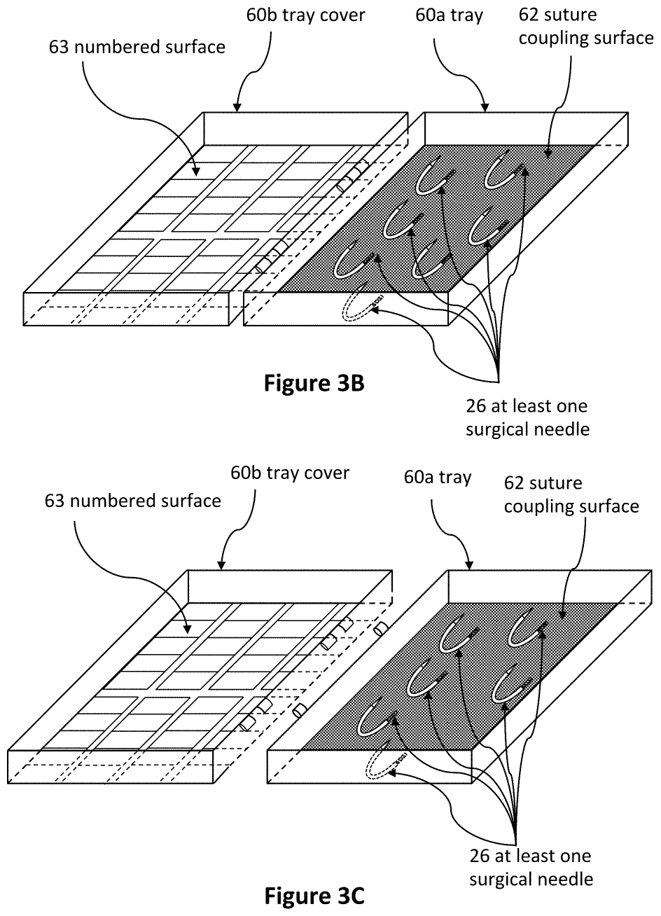

As illustrated in FIG. 3A, the system 10 may include a tray 60. The tray 60 may be arranged and configured to receive the at least one surgical needle 26. The tray 60 may be constructed of a translucent material, such as plexiglass, glass, plastic, rubber, silicone, any plastic like material, and the like. The tray 60 may have an adhesive surface 62. The adhesive surface 62 of the tray 60 may be constructed of silicone, rubber, acrylic, filled silicone, any material deemed appropriate by those skilled in the art, and the like. The adhesive surface 62 may compose a portion of the tray 60 that may come into contact with the at least one surgical needle 26, such that the at least one surgical needle 26 may be retained upon the adhesive surface 62 of the tray 60.

For example, the tray 60 may be carried by a member of a medical team during a medical procedure as the member of the medical team collects the at least one surgical needle 26 for counting by the system 10. The adhesive surface 62 of the tray 60 may ensure that the at least one surgical needle 26 does not fall off of the tray 60. This may save time during the surgical procedure, as in the case that the at least one surgical needle 26 falls off of the tray, members of the medical team may be required to spend additional time in the surgical environment looking for the at least one surgical needle 26. In many cases, time in the surgical environment is critical, as it can be costly and maintaining a patient's health may rely upon each moment spent in surgery.

In some embodiments, the tray 60 may be disposable. The tray 60 may be disposable to help support a sterile surgical environment, as the tray 60 may directly contact surgical items after such surgical items are used in a medical procedure. In some embodiments, a plurality of trays 60 may exist in the surgical field. For example, multiple members of the medical team may use the plurality of trays 60 to collect at least one of the at least one surgical needle 26. Each tray 60 may be positioned within the system 10 one at a time, and the system 10 may add the count of the at least one surgical needle 26 on each tray 60 to total the final quantity 30. The plurality of trays 60 may expedite the time to count the at least one surgical needle 26 and may thusly save time in the surgical environment, which, as earlier discussed, may be valuable. In some embodiments, the tray 60 may be re-usable. The re-usable tray 60 may have a removeable adhesive surface 62, such that the adhesive surface 62 may be disposed of with the at least one surgical needle 26 after the at least one surgical needle 26 has exited the surgical field and been accounted for by the system 10.

In some embodiments, the adhesive surface 62 of the tray 60 may define a magnetic surface. The magnetic surface of the tray 60 may be accomplished by incorporating at least one magnetic material into the tray 60. The at least one magnetic material may include at least one of iron, nickel, cobalt, magnesium, lithium, and the like. The magnetic material may comprise small magnetic pieces dispersed throughout a portion of the tray 60 with which the at least one surgical needle 26 may into contact. The magnetic material may also be embodied by at least one magnetic strip placed along the edge of the tray 60. The magnetic surface may be used with surgical items that may comprise magnetic properties, such as surgical needles, forceps, retractors, scalpels, clips, clamps, and the like.

With added reference to FIG. 3A, the bottom portion 52 of the stand 12 of the system 10 may be sized and configured to receive the tray 60. The tray 60 may carry the at least one surgical needle 26 when received within the stand 12. The camera 14 may be arranged and configured such that the camera 14 may be able to capture an image 28 of an entire surface of the tray 60 comprising the at least one surgical needle 26. The system 10 may be arranged and configured to automatically prompt the camera 14 to capture the image 28 of the tray 60 of the at least one surgical needle 26 upon detecting that the tray 60 of the at least one surgical needle 26 has been received within the stand 12. In some embodiments, the system 10 may be arranged and configured such that the camera 14 may capture the image 28 of the tray 60 of the at least one surgical needle 26 upon a user input on the touch screen device 16. The user may be able to control when the count occurs by interacting with a user interface of the touch screen device 16.

In some embodiments, the system 10 may include a backlight 64 located along the bottom portion 52 of the stand 12. The backlight 64 may be arranged and configured to provide contrast between the at least one surgical needle 26 and the tray 60. The translucent material comprising the tray 60 may allow light from the backlight 64 to partially pass through the tray 60. The light passing through the tray 60 may create a contrast between the light-bearing tray 60 and the opaque-colored nature of the at least one surgical needle 26. The contrast created between the tray 60 and the at least one surgical needle 26 may aid the system 10 in analyzing and counting the at least one surgical needle 26 on the tray 60.

FIGS. 3B and 3C illustrate an embodiment of the tray 60a. The tray 60a may include a tray cover 60b, wherein the tray 60a is removably coupled to the tray cover 60b. The tray 60a may be at least partially constructed of a magnetic material, such that the suture coupling surface 62 of the tray 60a may define a magnetic surface. The magnetic material may include at least one of iron, nickel, cobalt, magnesium, lithium, and the like. The magnetic surface may be used with surgical items that may comprise magnetic properties, such the at least one surgical needle 26, forceps, retractors, scalpels, clips, clamps, and the like.

In some embodiments, the tray cover 60b may comprise a numbered surface 63. The numbered surface 63 may be composed of a sponge-like material. The at least one surgical needle 26 may be poked into the sponge-like material of the tray cover 60b, wherein the at least one surgical needle 26 may couple to the tray cover 60b. At least one of the at least one surgical needle 26 may be coupled to a number on the numbered surface 63 of the tray cover 60b such that there is one at least one surgical needle 26 per number. The tray 60a and tray cover 60b may be folded together and the at least one surgical needle 26 may couple to the magnetic suture coupling surface 62. The system 10 may thus augment current manual surgical item counting practices to provide a second, reliable count of surgical items.

As illustrated in FIG. 3C, the tray 60a may be unattached from the tray cover 60b. The tray 60a may be placed within the stand 12 and the at least one surgical needle 26 may be electronically counted by the system 10. In some embodiments, the tray cover 60b may be folded underneath the tray 60a in order for the tray 60a to fit within the stand 12.

In some embodiments, the magnetic surface of the tray 60a may be accomplished by incorporating at least one magnetic material into the tray 60a. The at least one magnetic material may include at least one of iron, nickel, cobalt, magnesium, lithium, and the like. The magnetic material may comprise small magnetic pieces dispersed throughout a portion of the tray 60a with which the at least one surgical needle 26 may into contact. The magnetic material may also be embodied by at least one magnetic strip placed along the edge of the tray 60a. The magnetic surface may be used with surgical items that may comprise magnetic properties, such as the at least one surgical needle 26, forceps, retractors, scalpels, clips, clamps, and the like.

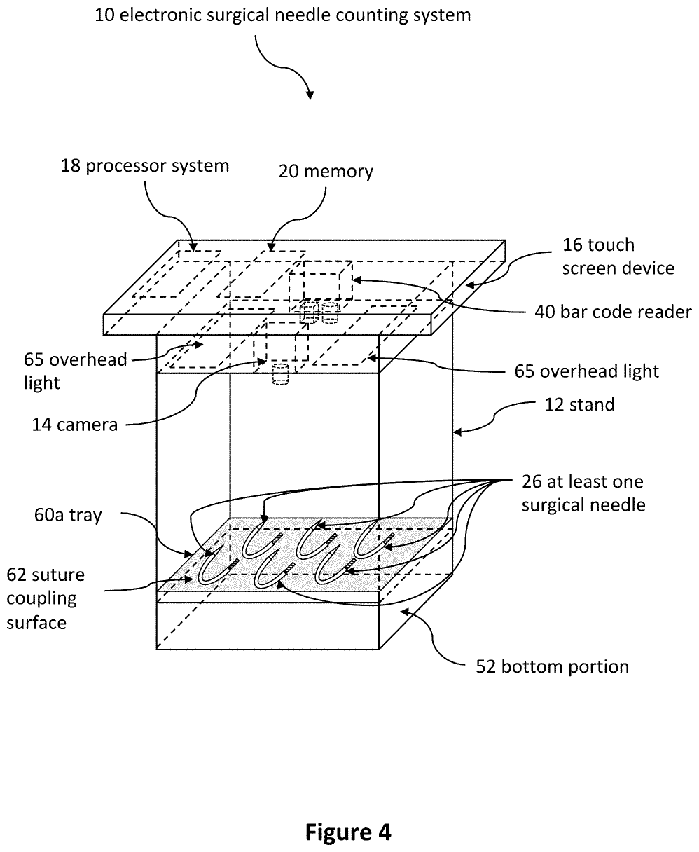

Referring now to FIG. 4, the system 10 may include an overhead light 65. The overhead light 65 may be positioned within the top portion 50 of the stand 12, such that the overhead light 65 may be next to the camera 14. In some embodiments, there may be a plurality of overhead lights 65 included in the system 10. The overhead light 65 may provide lighting from above the tray 60a in order to fully light the tray 60a so that the system 10 may capture and analyze an image of the at least one surgical needle 26. For example, if the tray 60a is constructed of a magnetic material, the backlight 64 may not provide proper lighting, as magnetic material is opaque. The overhead light 65 may provide lighting from above the tray 60a such that when the camera 14 captures an image of the at least one surgical needle 26, the system 10 may be able to analyze and differentiate each of the at least one surgical needle 26.

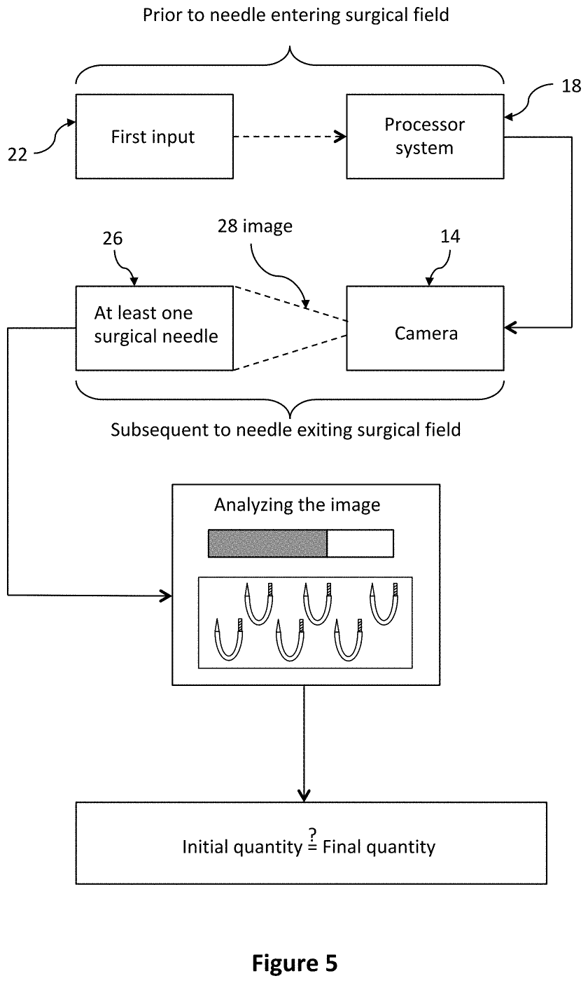

FIG. 5 illustrates a schematic of the operations that the processor system 18 may effectuate. Such operations may be effectuated by the processor system 18 based on executable instructions comprising the memory 20. The operations may include the processor system 18 receiving a first input 22. The first input 22 may consist of at least one of a style, dimension, type, curvature, body, point, shape, length, color, the initial quantity 24, and the like of the at least one surgical needle 26. The processor system 18 may receive the first input 22 prior to the at least one surgical needle 26 entering a surgical field.

The operations may also include the camera 14 capturing the image 28 of the at least one surgical needle 26. The camera 14 may capture the image 28 after at least one of the at least one surgical needle 26 has exited the surgical field. As well, the operations may include the processor system 18 analyzing the image 28. The processor system 18 may determine the final quantity 30 of the at least one surgical needle 26 after the at least one of the at least one surgical needle 26 has exited the surgical field based upon the analysis. Furthermore, the operations may include the processor system 18 determining whether the initial quantity 24 equals the final quantity 30.

In some embodiments, the camera 14 may capture a plurality of images 28 wherein a plurality of trays 60 is used to collect the at least one surgical needle 26 from the surgical field. The processor system 18 may analyze each of the plurality of images 28 to determine a quantity of the at least one surgical needle 26 on each tray 60, and add all of the quantities to determine the final quantity 30. Thus, the system 10 may begin accounting for the at least one surgical needle 26 before every at least one surgical needle 26 has been removed from the surgical field. Such systems may aid the medical team in accounting for the at least one surgical needle 26 during surgical procedures wherein multiple surgical needles are used and removed throughout the procedure. The medical team may not have to keep track of which of the at least one surgical needles 26 have been accounted for, but rather let the system 10 count the at least one surgical needle 26 and then dispose of the at least one surgical needle 26 and the tray 60 after counting, wherein the tray 60 is disposable.

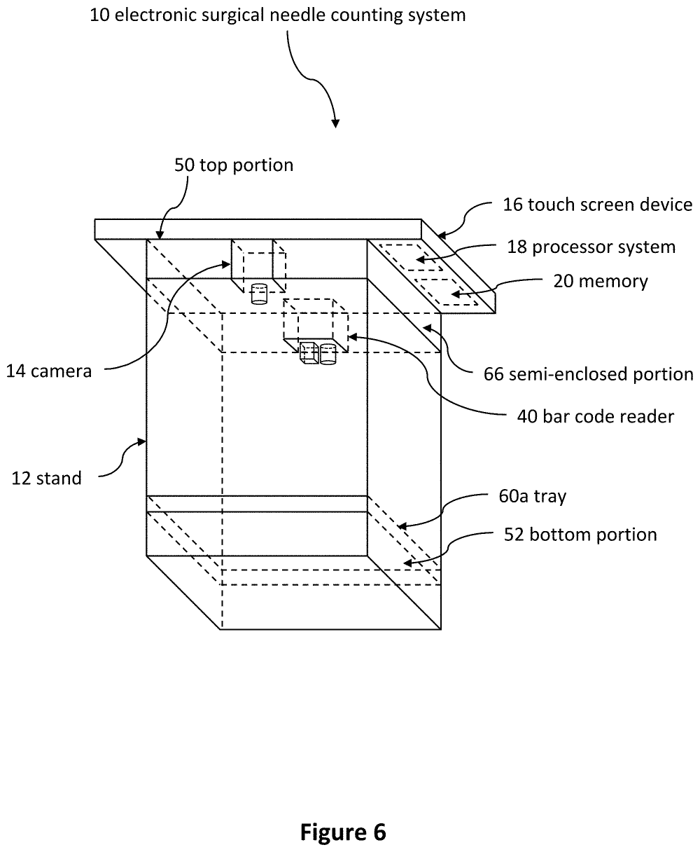

Referring now to FIG. 6, the system 10 may include the camera 14. The camera 14 may be located adjacent to the top portion 50 of the stand 12. The camera 14 may be vertically located between the touch screen device 16 and the bottom portion 52 of the stand 12. The system 10 may also include the bar code reader 40. The bar code reader 40 may be located adjacent to the top portion 50 of the stand 12. The bar code reader 40 may be vertically located between the touch screen device 16 and the bottom portion 52 of the stand 12.

In some embodiments, the stand 12 may incorporate a semi-enclosed portion 66. The semi-enclosed portion 66 may be vertically located between the top portion 50 and the bottom portion 52 of the stand 12. Furthermore, the camera 14 and the bar code reader 40 may be located within the semi-enclosed portion 66. The semi-enclosed portion 66 may be constructed of the same material as the stand 12. The semi-enclosed portion 66 may include openings in which the camera 14 and the bar code reader 40 may be positioned such that the camera 14 and the bar code reader 40 may be able to capture items that may be placed on the bottom portion 52, such as the tray 60 of at least one surgical needle 26 and a package of at least one surgical needle 26 that may include a bar code.

In many embodiments, the touch screen device 16 may incorporate the processor system 18 and the memory 20. The touch screen device 16 may be communicatively coupled with the processor system 18 and the memory 20. The memory 20 may comprise executable instructions that may be effectuated by the processor system 18. Such executable instructions and order thereof may be configurable to fit the needs of the user of the system 10. The touch screen device 16 may be communicatively coupled with the camera 14 such that when the camera 14 captures an image 28 of the at least one surgical needle 26 on the tray 60, the processor system 18 may analyze the image 28 and determine at least one of style, dimension, type, curvature, body, point, shape, length, color, the initial quantity 24, and the like of the at least one surgical needle 26.

The touch screen device 16 may also be communicatively coupled with the bar code reader 40 such that when the bar code reader 40 detects and reads a bar code, the processor system 18 may analyze information associated with the bar code. The system 10 may thus be enabled to gather information associated with the at least one surgical needle 26 when the at least one surgical needle 26 is within a package that may comprise the bar code. The information associated with the bar code may include at least one of style, dimension, type, curvature, body, point, shape, length, color, the initial quantity 24, and the like of the at least one surgical needle 26. The information associated with the bar code may be processed by the processor system 18 to constitute the first input 22.

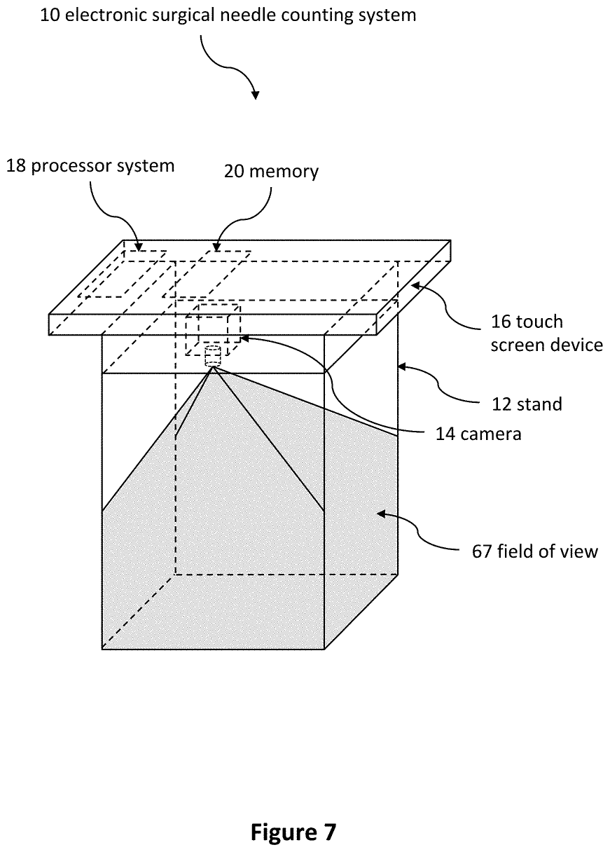

FIG. 7 illustrates a field of view 67 of the camera 14. The camera 14 may be positioned within the semi-enclosed portion 66 such that the camera 14 may capture an entire surface area of the tray 60 when the tray 60 is positioned within the stand 12 on the bottom portion 52. The stand 12 may be sized and configured to maintain an adequate focal length to allow the camera 14 to capture the image 28 of the entire surface area of the tray 60 of the at least one surgical needle 26. The stand 12 may also be sized and configured such that the field of view 67 of the camera 14 may encompass an area above the bottom portion 52 of the stand 12 on which the tray 60 is placed. This may allow for the camera 14 to capture the image 28 of the tray 60 wherein the tray 60 is placed a distance above the bottom portion 52. For example, the stand 12 may be arranged and configured to receive a plurality of trays 60, wherein the plurality of trays 60 may be placed above one another. In another example, surgical items with a height larger than the at least one surgical needle 26 may be counted within the system 10 without any part of the surgical items being cut out of the field of view 67 of the camera 14.

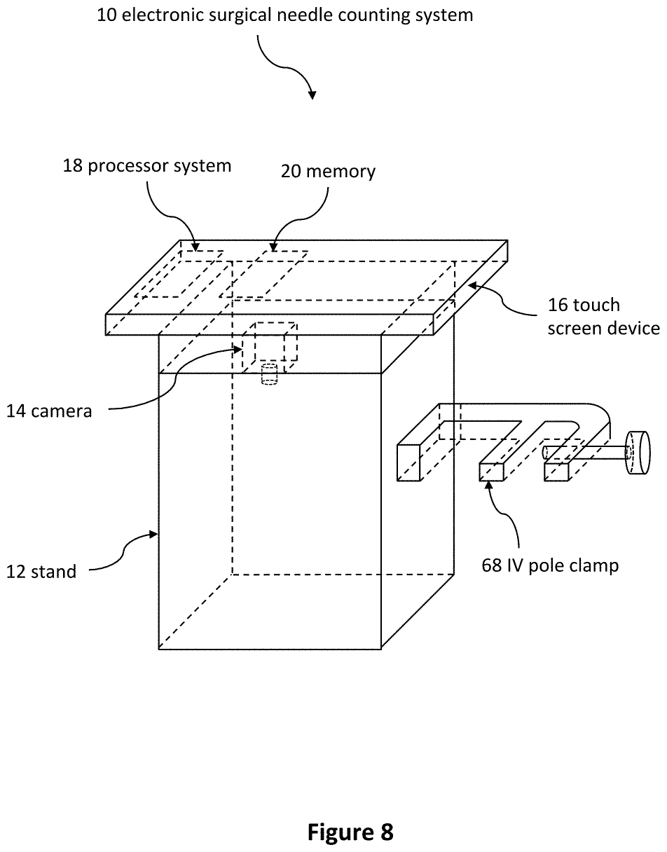

As illustrated in FIG. 8, the system 10 may include an IV pole clamp 68. The IV pole clamp 68 may be arranged and configured to couple the stand 12 to an IV pole. The ability of the stand 12 to couple to an IV pole may serve a variety of purposes during a surgical procedure. For example, if the system 10 is used during an emergency procedure, the operating area of the medical procedure may be small, and there may not be a space to set up the system 10 for use. Therefore, the IV pole clamp 68 coupled to the stand 12 may enable the system 10 to be used in a space saving manner within reach of the medical team, as the stand 12 may be coupled to an IV pole.

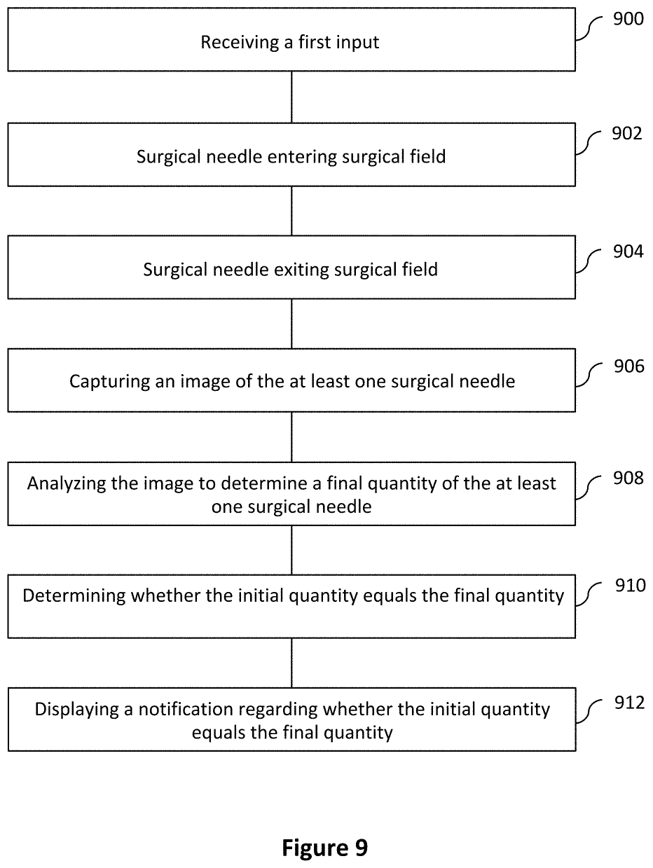

In some embodiments, the system 10 may consist of a stand 12, a camera 14 coupled to the stand 12, a touch screen device 16 communicatively coupled to the camera 14, a processor system 18 communicatively coupled to the camera 12, and a memory 20 communicatively coupled to the processor system. As illustrated in FIG. 9, methods for operating the system 10 may include receiving a first input 22 (at step 900). The first input 22 may consist of at least one of a style, dimension, type, curvature, body, point, shape, length, color, an initial quantity 24, and the like of the at least one surgical needle 26 prior to the at least one surgical needle 26 entering a surgical field (at step 902). After the at least one surgical needle 26 exiting the surgical field (at step 904), methods may further include capturing an image 28 of the at least one surgical needle 26 (at step 906). In response to capturing the image 28, methods may include analyzing the image 28 to determine a final quantity 30 of the at least one surgical needle 26 (at step 908). Methods may also include determining whether the initial quantity 24 equals the final quantity 30 (at step 910). As well, some methods may include displaying a notification on the touch screen device 16 regarding whether the initial quantity 24 equals the final quantity 30 (at step 912).

In some embodiments, a user of the system 10 may enter the first input 22 manually. The user may utilize a user interface incorporated in the touch screen device 16 to input the initial quantity 24 of the at least one surgical needle 26. In some embodiments, the user may utilize the user interface on the touch screen device 16 to adjust the initial quantity 24 of the at least one surgical needle.

In some embodiments, the system 10 may include a bar code reader 40. The bar code reader 40 may be coupled to the stand 12 and communicatively coupled to the touch screen device 16. According to some embodiments, methods may include the system 10 receiving the first input 22 (at step 900) via the bar code reader 40 prior to the at least one surgical needle 26 entering the surgical field (at step 902). The at least one surgical needle 26 may be contained within a package prior to use, the package comprising a bar code. The bar code reader 40 may read the bar code of the package such that the processor system 18 receives information regarding the at least one surgical needle 26, such as the initial quantity 24. The processor system 18 of the touch screen device 16 may comprise a software program that may have a built-in database of needle styles and dimensions. The processor system 18 may analyze the bar code of a package of at least one surgical needle 26 and retrieve information from said database to receive the first input 22.

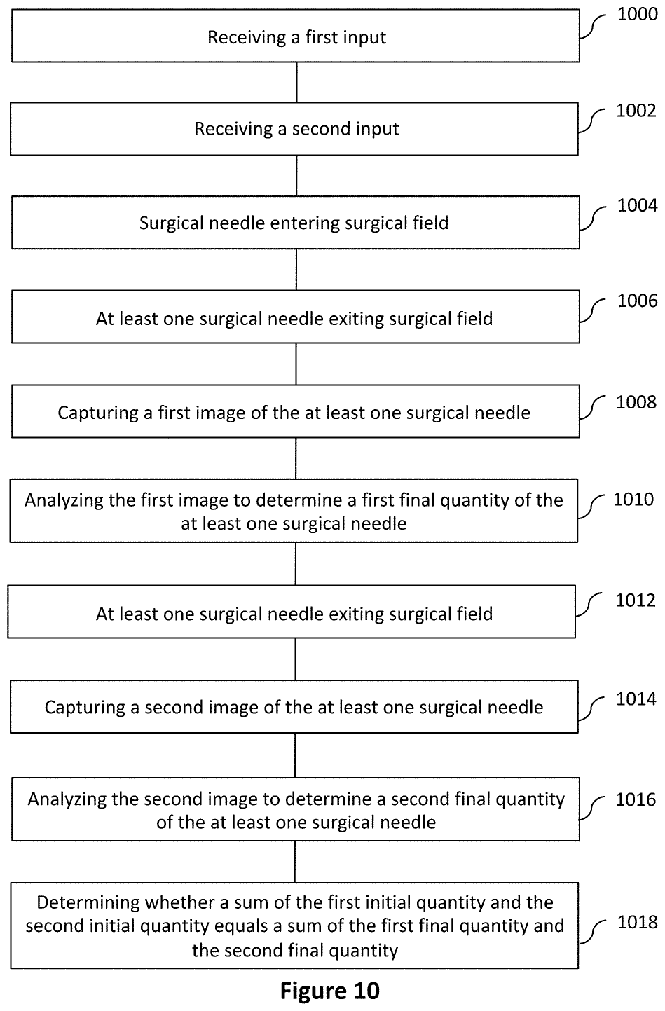

FIG. 10 illustrates a method that includes the system 10 receiving a plurality of initial inputs. Methods may include the system 10 receiving the first input 22 (at step 1000). The first input may define a first initial quantity 74. Methods may also include the system 10 receiving a second input 72 (at step 1002). The second input 72 may include at least one of the style, dimension, type, curvature, body, point, shape, length, color, a second initial quantity 76, and the like of the at least one surgical needle 26. The system 10 may receive the first input 22 and the second input 72 prior to the at least one surgical needle 26 entering the surgical field (at step 1004). After at least one of the at least one surgical needle 26 exiting the surgical field (at step 1006), methods may further include the camera 14 capturing a first image 70 of the at least one surgical needle 26 (at step 1008). In response to the camera 14 capturing the first image 70, methods may include the processor system 18 analyzing the first image 70 to determine a first final quantity 80 of the at least one surgical needle 26 (at step 1010).

With continued reference to FIG. 10, some methods may include the system 10 capturing a second image 78. After at least one of the at least one surgical needle 26 exiting the surgical field (at step 1012), methods may include the camera 14 capturing the second image 78 of the at least one surgical needle 26 (at step 1014). In response to the camera 14 capturing the second image 78, methods may include the processor system 18 analyzing the second image 78 to determine a second final quantity 82 of the at least one surgical needle 26 (at step 1016). Methods may further include the processor system 18 determining whether a sum of the first initial quantity 74 and the second initial quantity 76 equals a sum of the first final quantity 80 and the second final quantity 82 (at step 1018).

In some embodiments, methods may include displaying a notification on the touch screen device 16. The notification may be regarding whether the sum of the first initial quantity 74 and the second initial quantity 76 equals the sum of the first final quantity 80 and the second final quantity 82. In some embodiments, the touch screen device 16 may operate on a processor system 18 available to people outside of the operating room, such that the notification may appear on devices outside of the operating room operating on the processor system 18.

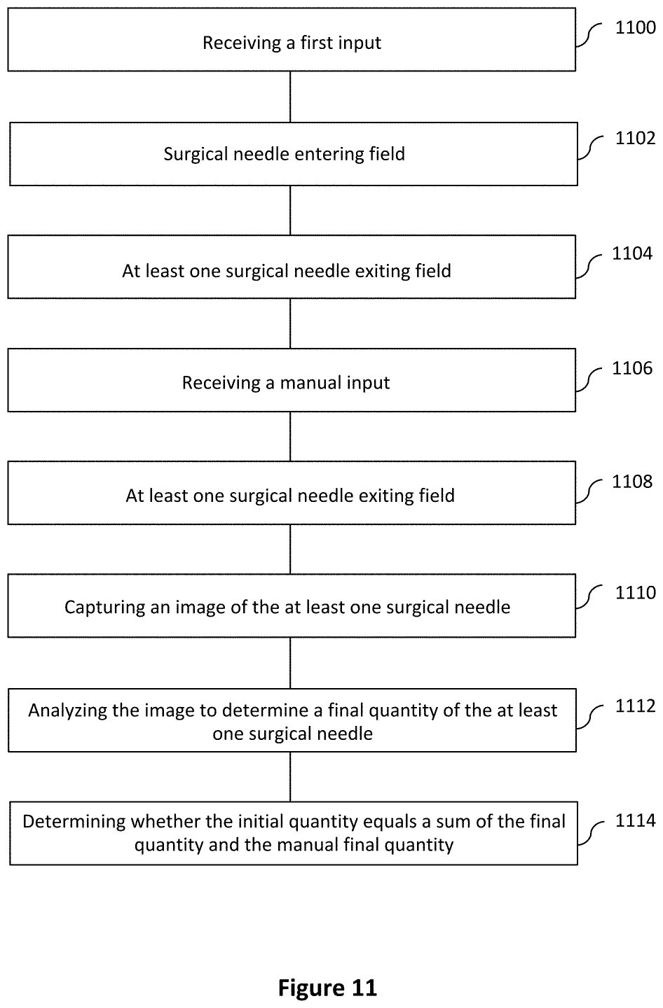

Furthermore, as shown in FIG. 11, some methods may include using the system 10 in combination with manual surgical needle counting systems. Methods may include the system 10 receiving the first input 22 (at step 1100). The first input 22 may consist of at least one of a style, dimension, type, curvature, body, point, shape, length, color, an initial quantity 24, and the like of the at least one surgical needle 26. The system 10 may receive the first input 22 prior to the at least one surgical needle 26 entering the surgical field (at step 1102). After at least one of the at least one surgical needle 26 exiting the surgical field (at step 1104), methods may include the touch screen device 16 receiving a manual input 84 (at step 1106). The manual input 84 may include a manual final quantity 86 of the at least one surgical needle 26. The manual input 84 may be received by the system 10 while at least one of the at least one surgical needle 26 is still located on the surgical field.

With continued reference to FIG. 11, after the at least one surgical needle 26 exiting the surgical field (at step 1108), methods may include the camera 14 capturing the image 28 of the at least one surgical needle 26 (1110). In response to the camera 14 capturing the image 28, methods may also include the processor system 18 analyzing the image 28 to determine a final quantity 30 of the at least one surgical needle 26 (at step 1112). Methods may further include the processor system 18 determining whether the initial quantity 24 equals a sum of the final quantity 30 and the manual final quantity 86 (at step 1114).

In some embodiments, the touch screen device 16 of the system 10 may comprise software that may possess the ability to log surgery time and various counts of surgical items and store such information in the memory 20. The system 10 may also be able to collect a patient number, as well as any employee information related to members of a medical team involved in a surgical procedure. Such information may be gathered by the system 10 by methods including scanning an employee identification card using the bar code reader 40, using the user interface to enter information, and the like. The processor system 18 may also be able to communicate with at least one of patient charting software and printers using at least one of wireless networking and application programming interfaces.

### Interpretation

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1 and embodiments described in other sections may apply to and be combined with embodiments described within the “Topic 1” section.

Some of the devices, systems, embodiments, and processes use computers. Each of the routines, processes, methods, and algorithms described in the preceding sections may be embodied in, and fully or partially automated by, code modules executed by one or more computers, computer processors, or machines configured to execute computer instructions. The code modules may be stored on any type of non-transitory computer-readable storage medium or tangible computer storage device, such as hard drives, solid state memory, flash memory, optical disc, and/or the like. The processes and algorithms may be implemented partially or wholly in application-specific circuitry. The results of the disclosed processes and process steps may be stored, persistently or otherwise, in any type of non-transitory computer storage such as, e.g., volatile or non-volatile storage.

The various features and processes described above may be used independently of one another, or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, in parallel, or in some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments include A, B, and C. The term “and/or” is used to avoid unnecessary redundancy.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

The following is claimed:

1. A surgical needle counting system, comprising:
a stand;
a camera coupled to the stand;
a touch screen device communicatively coupled to the camera;
a processor system communicatively coupled to the camera; and
memory communicatively coupled to the processor system, the memory comprising executable instructions that, when executed by the processor system, cause the processor system to effectuate operations comprising:
receiving a first input comprising at least one of a style, dimension, and an initial quantity of at least one surgical needle prior to the at least one surgical needle entering a surgical field;
capturing an image of the at least one surgical needle after at least one of the at least one surgical needle has exited the surgical field;
analyzing the image to determine a final quantity of the at least one surgical needle after the at least one of the at least one surgical needle has exited the surgical field; and
determining whether the initial quantity equals the final quantity.

2. The surgical needle counting system of claim 1, further comprising a bar code reader coupled to the stand and communicatively coupled to the touch screen device.

3. The surgical needle counting system of claim 2, wherein the stand defines a rectangular shape having a first sidewall, a second sidewall opposite the first sidewall, a third sidewall adjacent the first and second sidewalls, an open side opposite the third sidewall, a top portion, and a bottom portion opposite the top portion.

4. The surgical needle counting system of claim 3, wherein the stand is constructed of a translucent material.

5. The surgical needle counting system of claim 4, wherein the touch screen device is coupled to the top portion of the stand.

6. The surgical needle counting system of claim 5, further comprising a tray arranged and configured to receive the at least one surgical needle, the tray being translucent and having an adhesive surface.

7. The surgical needle counting system of claim 6, wherein the bottom portion of the stand is sized and configured to receive a tray of surgical needles, and wherein the camera is arranged and configured so that the camera is able to capture images of an entire surface of the tray of surgical needles.

8. The surgical needle counting system of claim 7, further comprising a backlight located along the bottom portion such that the backlight is arranged and configured to provide contrast between the at least one surgical needle and the tray.

9. The surgical needle counting system of claim 5, wherein the camera is located adjacent the top portion of the stand and vertically located between the touch screen device and the bottom portion.

10. The surgical needle counting system of claim 9, wherein the bar code reader is located adjacent the top portion of the stand and vertically located between the touch screen device and the bottom portion.

11. The surgical needle counting system of claim 10, wherein the stand comprises a semi-enclosed portion vertically located between the top portion and the bottom portion, and wherein the camera and the bar code reader are located within the semi-enclosed portion.

12. The surgical needle counting system of claim 1, wherein the processor system and memory are integrated into the touch screen device.

13. The surgical needle counting system of claim 1, further comprising an IV pole clamp coupled to the stand, the IV pole clamp arranged and configured to couple the stand to an IV pole.

14. A method of counting surgical needles using a surgical needle counting system comprising a stand, a camera coupled to the stand, a touch screen device communicatively coupled to the camera, a processor system communicatively coupled to the camera, and memory communicatively coupled to the processor system, the method comprising:
capturing, via the camera, a first image of the at least one surgical needle after at least one of the at least one surgical needle has exited the surgical field;
analyzing, via the processor system, the first image to determine a final quantity of the at least one surgical needle after the at least one of the at least one surgical needle has exited the surgical field; and
determining, via the processor system, whether the initial quantity equals the final quantity.

15. The method of counting surgical needles of claim 14, wherein the surgical needle counting system comprises a bar code reader coupled to the stand and communicatively coupled to the touch screen device, the method further comprising receiving, via the bar code reader, a first input comprising at least one of a style, dimension, and an initial quantity of at least one surgical needle prior to the at least one surgical needle entering the surgical field.

16. The method of counting surgical needles of claim 15, further comprising displaying, via the touch screen device, a notification regarding whether the initial quantity equals the final quantity.

17. The method of counting surgical needles of claim 16, wherein the initial quantity is a first initial quantity, the method further comprising receiving, via the bar code reader, a second input comprising at least one of the style, dimension, and a second initial quantity of the at least one surgical needle prior to the at least one surgical needle entering the surgical field.

18. The method of counting surgical needles of claim 17, further comprising capturing, via the camera, a second image of the at least one surgical needle after at least one of the at least one surgical needle has exited the surgical field.

19. The method of counting surgical needles of claim 18, wherein the final quantity is a first final quantity, the method further comprising:
analyzing, via the processor system, the second image to determine a second final quantity of the at least one surgical needle after the at least one of the at least one surgical needle has exited the surgical field; and
determining, via the processor system, whether a sum of the first initial quantity and the second initial quantity equals a sum of the first final quantity and the second final quantity.

20. The method of counting surgical needles of claim 16, further comprising:
receiving, via the touch screen, a manual input comprising a manual final quantity of the at least one surgical needle while the at least one surgical needle is still located on the surgical field; and
determining, via the processor system, whether the initial quantity equals a sum of the final quantity and the manual final quantity.
